(19) United States
(12) Patent Application Publication
(10) Pub. No.: US 2006/0104915 A1
(43) Pub. Date: May 18, 2006

(54) Aerosol dentifrice formulation

(76) Inventor: Peter Frost, Weybridge, Surrey (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/522,056

(22) PCT Filed: Jul. 22, 2003
(86) PCT No.: PCT/GB03/03150

§ 371 Date: Aug. 15, 2005

(30) Foreign Application Priority Data
Jul. 23, 2002 (GB) 0217078.5
Apr. 17, 2003 (GB) 0308978.6

(51) Int. Cl. A61K 8/19; A61K 9/14

(52) U.S. Cl. 424/46; 424/49

(57) ABSTRACT

An aerosol dentifrice formulation is provided comprising a high water based system, a specific particle size abrasive and a liquified gas propellant, characterised in that the liquified gas comprises 2-8 wt % of a non-hydrocarbon and 2-6 wt % of a hydrocarbon propellant. There is provided a dentifrice formulation comprising a fluid mixture which includes a liquified gas propellant and containing a particulate abrasive, characterised in that the particulate abrasive comprises 1-10% by weight of the mixture, has a particle size in the range 5-40 microns and comprises a combination of a more hard and a less hard abrasive. Another aspect of the invention provides for an aerosol dentirice comprising water, a particulate abrasive and a liquefied gas propellant, characterised in that the propellant comprises 2-8 wt % of a non-hydrocarbon and 2-6 wt % of a liquified hydrocarbon propellant, the particulate abrasive comprising 1-10% by weight of the mixture, has a particle size in the range 5-40 microns and comprises a combination of a more hard and a less hard abrasive.

This invention relates to dentifrice formulations, in particular to a dentifrice formulation that can be stored in a pressurised container from which it can be dispensed onto a toothbrush as a stable and controllable foam.

Dentifrices are commonly provided as a paste, i.e. a toothpaste, in a collapsible container from which they can be extruded. It is also known to provide a dentifrice formulation as a foam, e.g. as disclosed in DE-A-100 08 834, DE-A-100 08 836 and DE-A-100 08 837. Such formulations generally comprise a fluid mixture containing one or more abrasive, thickener, flavour etc. together with a propellant, normally a liquefied gas with a boiling point below ambient temperature to drive the formulation out of its container and to expand to foam the formulation. The propellant in these prior art formulations will produce a post-foaming effect.

With this type of formulation it is particularly important to provide a foaming dentifrice that is both practical in use but also gives an immediate visual impact upon dispensing. Japanese Patent Application No. 51-12593B discloses a dual phase oil-in-water emulsion formulation for a rapidly collapsing foam aerosol product. The formulation comprises a base agent and a mixed propellant comprising 70-95 wt % dimethylether (DME) and 30-5 wt % water insoluble liquified gas such as a flurocarbon, liquid petroleum, vinyl chloride and methyl chloride. This propellant system is important for products that require a rapid collapsing foam like nail polish or hair spray.

With aerosol dentifrice formulations it is very important to control the production and expansion of foam so that it can be controlled, particularly onto a small surface area toothbrush head. Also, the post-drooling effect on and around the actuator suffered by many aerosol products can lead to unattractive product that becomes unacceptable to use. It is important therefore that the product must not expand significantly further once dispensed onto a toothbrush or suffer from post-drooling effects. With this control and aesthetic appeal the product will be both pleasant and acceptable to the user.

Dentifrice formulations usually contain a high proportion of water and many of the more commonly used propellants, eg, hydrocarbon propellants, are not very miscible with water making the formulation of an acceptable foaming dentifrice product a challenge for those skilled in the art. As a result, many propellants will not provide acceptable foaming dentifrice formulations, particularly those formulations that are single phase water based.

Therefore it is an object of the present invention to provide a foaming dentifrice formulation with a suitable propellant system that produces a very stable foam and avoids all of the above mentioned problems.

The word foam in the text means a viscous liquid material that includes a large number of small bubbles of vapour of a propellant dispersed therein.

According to this invention an aerosol dentifrice formulation is provided comprising water, a particulate abrasive and a propellant, characterised in that the propellant comprises 2-8 wt % of a non-hydrocarbon and 2-6 wt % of a hydrocarbon propellant

In a further aspect of the invention there is provided an aerosol dentifrice formulation being a fluid mixture, comprising water, a liquified gas propellant and a particulate abrasive, characterised in that the particulate abrasive comprises 1-10% by weight of the mixture, has a particle size in the range 5-40 microns and comprises a combination of a more hard and a less hard abrasive.

A third aspect of the invention provides an aerosol dentifrice comprising water, a particulate abrasive and a liquefied gas propellant, characterised in that the propellant comprises 2-8 wt % of a non-hydrocarbon and 2-6 wt % of a hydrocarbon propellant, the particulate abrasive comprising 1-10% by weight of the mixture, has a particle size in the range 5-40 microns and comprises a combination of a more hard and a less hard abrasive.

Suitable liquified gas propellants include non-hydrocarbon and hydrocarbon propellants. These propellants are well known to those skilled in the art of aerosol formulation.

Suitable non-hydrocarbon propellants include dimethylether (DME), chlorofluorocarbon (CFC) hydrofluorocarbon (HFC), hydrochlorofluorocarbon (HCFC) such as nitrogen, carbon dioxide, nitrous oxide and compressed air. A preferred non-hydrocarbon propellant is DME. DME is useful as a propellant because soluble in water based products.

Suitable hydrocarbon propellants generate a range of pressures of ca. 16-105 psi. Many propellants are known which can achieve this, suitably a commercial product “Butane 30” comprising a mixture of n-butane, i-propane and n-propane. Experiments have shown that when the dentifrice formulation comprises a mixture of hydrocarbon propellant such as n-butane and non-hydrocarbon propellant DME an optimised product is achieved that has a particularly stable foam without any significant expansion or collapsing of the foam.

Suitable liquified hydrocarbon propellants include one or more C₃ to C₅ hydrocarbon (HC) such as propane, n-butane or butane 22.

Typically a maximum total of 8 wt % propellant is used, more preferably 5 wt %. Preferably the dentifrice formulation of the present invention may comprise between 2-5 wt % of such a liquefied gas propellant.

The formulation is normally stored in a container provided with a release valve, under a pressure corresponding to the vapour pressure of the liquefied propellant at the storage temperature, and on opening the valve the formulation is expelled as a foam, e.g. onto a toothbrush head.

Typically the container may be provided with an actuator device by means of which the valve can be opened and the flow of formulation directed.

The abrasive typically comprises 1-10% by weight of the total mixture and has a particle size in the range of between 5-40 microns. Preferably the formulation comprises 9 wt % or less, e.g. 3-7 wt % abrasive, especially 4.5-6 wt %, typically ca. 5 wt %. The abrasive particle size is in the range of 5-40 microns, preferably 30 microns or less, more preferably 10 microns or less. Suitably a mixture of at least one less hard and at least one more hard particulate abrasive is used, typically in a proportion more hard: less hard in the range 1:1-5, suitably in the range 1:2.5-3.5. Suitably the abrasive material may be a silica or a combination of silicas. Less hard and more hard abrasives can also be called soft and hard abrasives and this will refer specifically to the hardness of the abrasive particle. Suitable silicas include those known as Zeodent 124™ (hard) and Zeodent 623™ (soft). The proportion and particle size of the abrasive are found to optimise the combination of suitability for flow of the formulation out through the valve and effective tooth cleaning.

The formulation preferably also contains one or more of the following.

One or more humectant, typically in a proportion of 25-75 wt %, preferably 45-55 wt %, especially 50±2 wt %. Humectants are added to protect the formulation from drying out and to provide consistency and protection against cold. Suitable humectants include sorbitol and glycerol. Suitably a mixture of sorbitol and glycerol may be used e.g. in a sorbitol:glycerol ratio in the range 1:1.5-1.5:1. Other humectants that may be used include xylitol, mannitol, 1,2-propylene glycol or mixtures of these polyols.

One or more slurrying/suspending agent, typically in a proportion of 1-5 wt %, preferably 2-3 wt %. A preferred slurrying agent is polyethylene glycol, e.g. of molecular weight in the range 200-800, typically ca. 300.

One or more surfactant. Typically a surfactant may be used as a foaming agent as foaming is one property of a surfactant. In this respect a surfactant is sometimes called a foaming agent. Suitable surfactants include anionic surfactants such as a sodium alkyl sulphate with a 12-18 carbon atoms in the alkyl chain, such as sodium lauryl sulphate. Zwitterionic, ampholytic and nonionic surfactants may also be used. A mixture of surfactants may be used. Suitably the surfactant may comprise 0.1-3.0 wt % of the formulation, preferably 1-2 wt %.

One or more thickening agent. Typically a thickening agent will add body to the foam. Typical thickening agents include hydroxypropylmethylcellulose (HPMC), hydtrxyethylcellulose (HEC) and hydroxymethylcellulose (HMC) and the acrylic polymer Carbopol. Preferred thickening agents include xanthan gum which is a polysaccharide and/or a thickening silica, Zeodent 163. Typically the thickening agent may comprise 0.1-4.0 wt % of the formulation, typically 0.2-3.0 wt %. It is found that use of xanthan gum and Zeodent 163 can lead to a creamier foam with improved flow and texture characteristics.

One or more pH regulator, preferably to maintain the pH at 6.0-10.0, especially at ca. pH 8.0. Such a pH is found suitable to avoid corrosion of the tin plate, aluminum or containers that may be lacquered that are commonly used for containing such formulations. A suitable pH regulator is sodium hydroxide.

One or more other excipent such as a sweetener, colour, preservative, flavours, dyes etc., typically comprising up to ca. 2 wt % of the formulation.

Such compositions of the present invention may also comprise other active agents conventionally used in dentifrice compositions, for instance: an antimicrobial agent, anti-plaque agent such as chlorhexidine or triclosan; anti-calculus agent such as a tetra- or a di-alkali metal pyrophosphate salt, or a mixture thereof, an alkali metal tripolyphosphate salt or an azacycloheptane diphosphonate salt; an anti-sensitivity agent that acts as a nerve depolariser, tubule occluder or mineralizer. These agents include e.g. strontium acetate, strontium chloride or a potassium salt such as potassium nitrate, potassium chloride or potassium citrate; remineralisation agent, a whitening agent such as tetra- or a di-alkali metal pyrophosphate or phosphate salts or peroxides, vitamins, fluoride, e.g. sodium fluoride, typically comprising up to ca. 0.5 wt % of the formulation. Such agents will be included at levels to provide the desired therapeutic effect.

Many other examples of materials of these types are known in the state of the art, e.g. in DE-A-100 08 837, the content of which is incorporated herein by way of example only.

In order for the formulation to be a fluid mixture, the remainder of the formulation comprises water, typically the formulation comprises ca. 25-50 wt % water, preferably 30-40 wt % of the formulation.

A typical formulation according to this invention therefore comprises: one or more humectant 45-55 wt %, slurrying agent 2-3 wt %, surfactant 1-2 wt %, abrasives 3-7 wt %, preferably 3-5 wt %, thickening agent 0.2-0.5 wt %, flavour, active and sweetener 0-2 wt %, pH adjuster if necessary to provide pH of 8.5±0.2, water 30-40 wt % preferably 35±1 wt %. This fluid formulation which is also known as the intermediate formulation is preferably charged into a metal or plastic container with a dispensing valve, at a proportion of 95-97 wt % with 3-5 wt % propellant providing a pressure of between 25-70, preferably 40-60 psi.

The preferred materials and their proportions described above also contribute to improved flow and handling of the formulation.

The following observations in the finished product were noted:

| | | | | | |
|---|---|---|---|---|---|
| N-butane | 0% | 2% | 2.5% | 3% | 5% |
| Dimethylether | 5% | 3% | 2.5% | 2% | 0% |
| Observation | Foam | Stable | Very | Slightly | Very |
| | which | foam, no | slightly | expanding | expand- |
| | collapses | expansion | expanding | foam | ing |
| | instantly | or | foam | | foam |
| | | collapse | | | |

A “very slightly expanding foam” can be defined as a foam that when dispensed onto a toothbrush head may show a small but insignificant expansion.

A typical process for making the formulation of this invention may involve the steps of:
1. Adding a suitable quantity of water to a mixing vessel.
2. Adding sweetener and active to the water and agitating until dissolved or suspended.
3. Adding the humectant and agitating until homogeneous.
4. Sieving the abrasive to break up any lumps. A 500 micron sieve is generally suitable.
5. Slowly adding the abrasive to the mixture while mixing.
6. Slurrying the thickening agent and slurrying agent and add to the mixture, agitate until homogeneous.
7. Mixing, optionally transferring to a mixer.
8. Slurrying the flavour, dye and foaming agent and adding to the mixture, mixing until homogeneous.
9. Adjusting the pH and
10. Mixing until homogeneous.

This fluid mixture may then be charged into suitable valved containers together with a suitable quantity of propellant.

The formulation may be used in a generally conventional manner involving opening the valve of the container to allow the internal pressure to expel the formulation onto a toothbrush. The invention also provides a valved container containing a formulation as described above.

The invention will now be described by way of examples.

### EXAMPLE 1

A dentifice formulation was prepared having the following composition:

| Function | Component | wt % |
|---|---|---|
| Humectant | Sorbitol 70% non-crystallizing | 28.000 |
| Humectant | Glycerin | 22.00 |
| Suspending agent | PEG 6 | 2.500 |
| Foaming agent | Empicol 0303 30% solution | 5.000 |
| Sweetener | Sodium saccharin | 0.300 |
| Active | Sodium fluoride | 0.306 |
| Flavour | Flavour | 1.000 |
| Abrasive (hard) | Zeodent 124* | 1.330 |
| Abrasive (soft) | Zeodent 623* | 3.670 |
| Thickener | Xanthan (Keltrol F) | 0.250 |
| pH adjuster | 35% NaOH solution | 0.250 |
| Dye | Dye | 0.003 |
| Water | | to 100.000 |
| Propellant | Butane | 2.000 |
| Propellant | DME | 3.000 |

*Zeodent 124 and Zeoent 623 are trade names of Huber Corporation, USA

### EXAMPLE 2

A dentifice formulation was prepared having the following composition:

| Function | Component | wt % |
|---|---|---|
| Humectant | Sorbitol 70% non-crystallizing | 28.000 |
| Humectant | Glycerin | 22.00 |
| Suspending agent | PEG 6 | 2.500 |
| Foaming agent | Empicol 0303 30% solution | 5.000 |
| Sweetener | Sodium saccharin | 0.300 |
| Active | Sodium fluoride | 0.306 |
| Flavour | Flavour | 1.000 |
| Abrasive (hard) | Zeodent 124 | 1.330 |
| Abrasive (soft) | Zeodent 623 | 3.670 |
| Thickener | Zeodent 163 | 3.000 |
| pH adjuster | 35% NaOH solution | 0.250 |
| Dye | Dye | 0.003 |
| Water | | to 100.000 |
| Propellant | Butane | 3.000 |
| Propellant | DME | 2.000 |

*Zeodent 124 and Zeoent 623 and Zeodent 163 are trade names of Huber Corporation, USA

### EXAMPLE 3

A dentifice formulation was prepared having the following composition:

| Function | Component | wt % |
|---|---|---|
| Humectant | Sorbitol 70% non-crystallizing | 28.000 |
| Humectant | Glycerin | 22.00 |
| Suspending agent | PEG 6 | 2.500 |
| Foaming agent | Empicol 0303 solid | 1.500 |
| Sweetener | Sodium saccharin | 0.300 |
| Active | Sodium fluoride | 0.306 |
| Flavour | Flavour | 1.000 |
| Abrasive (hard) | Zeodent 124 | 1.330 |
| Abrasive (soft) | Zeodent 623 | 3.670 |
| Thickener | Zeodent 163 | 3.000 |
| Thickener | Xanthan (Keltrol F) | 0.250 |
| Dye | Dye | 0.003 |
| Water | | to 100.000 |
| Propellant | Butane | 3.000 |
| Propellant | DME | 2.000 |

*Zeodent 124 and Zeoent 623 and Zeodent 163 are trade names of Huber Corporation, USA

This fluid formulation was made by a process as described above, involving
1. Adding a suitable quantity of water to a mixing vessel.
2. Adding sweetener and active to the water, mixing until dissolved using a circular Heidolph paddle stirrer.
3. Adding the glycerol and sorbitol to the batch, mixing until dissolved using a circular paddle stirrer on the Heidolph.
4. Sieving the abrasive to break up any lumps. A 500 micron sieve is generally suitable.
5. Slowly adding the abrasive to the mixture, mixing using a circular paddle stirrer on the Heidolph.
6. Slurrying the thickening agent and slurrying agent and add to the mixture, agitate until homogeneous.
7. Transferring to an Ultra Turrux mixer and mixing for 5 minutes.
8. Slurrying the flavour and foaming agent and adding to the mixture, mixing until homogeneous with a circular paddle stirrer on the Heidolph.
9. Adjusting the pH to pH 8 (±0.5) using the NaOH.
10. Mixing until homogeneous on the Heidolph.

This fluid mixture was charged into valved containers together with the propellant in a 95:5 or 97:3 w/w ratio.

1. An aerosol dentifrice formulation comprising water, a particulate abrasive and a propellant, characterised in that the formulation contains 4-14 wt % of a propellant which comprises a non-hydrocarbon being 2-8 wt % of the formulation and a hydrocarbon propellant being 2-6 wt % of the formulation.

2. An aerosol dentifrice according to claim 1 wherein the non-hydrocarbon propellant comprises dimethylether (DME), a chlorofluorocarbon (CFC), a hydrofluorocarbon (HFC) or a hydrochlorofluorocarbon (HCFC), nitrogen, carbon dioxide, nitrous oxide or compressed air.

3. An aerosol dentifrice according to claim 2 wherein the non-hydrocarbon propellant comprises dimethylether (DME).

4. An aerosol dentifrice according to claim 1 wherein the hydrocarbon propellant comprises one or more C₃ to C₅ hydrocarbon (HC) such as propane, n-butane or butane 22.

5. An aerosol dentifrice according to claim 1 which comprises 3-5 wt % propellent providing-pressure of between 25-70 psi.

6. An aerosol dentifrice formulation being a fluid mixture, comprising water, a liquified gas propellant and a particulate abrasive, characterised in that the particulate abrasive comprises 1-10% by weight of the mixture and has a particle size in the range 5-40 microns and comprises a combination of a more hard and a less hard abrasive.

7. An aerosol dentifrice according to claim 6, wherein the more hard and less hard abrasive is in a proportion of 1:1-5.

8. An aerosol dentifrice according to claim 1, wherein the particulate abrasive is a silica.

9. An aerosol dentifrice according to claim 8, wherein the particle size of the abrasive is less than 30 microns.

10. An aerosol dentifrice according to claim 9, wherein the particle size of the abrasive is less than 10 microns.

11. An aerosol dentifrice comprising water, a particulate abrasive and a liquified gas propellant, characterised in that the particulate abrasive comprises 1-10% by weight of the mixture, has a particle size in the range 5-40 microns and comprises a combination of a more hard and a less hard abrasive.

12. An aerosol dentifrice according to claim 1 wherein the water comprises 25-50 wt % of the formulation.

13. An aerosol dentifrice according to claim 12, additionally comprising a humectant.

14. An aerosol dentifrice according to claim 13, additionally comprising a suspending agent.

15. An aerosol dentifrice according to claim 14, additionally comprising a surfactant.

16. An aerosol dentifrice according to claim 1 additionally comprising xanthan gum and Zeodent 163.

17. An aerosol dentifrice according to claim 1 comprising 45-55 wt % humectant, 0.1-4 wt % suspending agent, 1-5 wt % surfactant, 3-7 wt % abrasive, the remainder being water and 3-5 wt % propellant.

18. An aerosol dentifrice according to claim 1 comprising a valved container containing the formulation.
